(19) United States
(12) Patent Application Publication
(10) Pub. No.: US 2024/0382490 A1
(43) Pub. Date: Nov. 21, 2024

(54) QUINAZOLINE-THIOHYDANTOIN FUSED HETEROCYCLES WHICH ARE SUITABLE FOR TREATING, AMELIORATING OR PREVENTING A PROLIFERATIVE DISORDER

(71) Applicants: FRIEDRICH-ALEXANDER-UNIVERSITÄT ERLANGEN NÜRNBERG, Erlangen (DE); JOHANNES GUTENBERG-UNIVERSITÄT MAINZ, Mainz (DE)

(72) Inventors: Svetlana TSOGOEVA, Erlangen (DE); Thomas EFFERTH, Heidelberg (DE)

(21) Appl. No.: 18/683,648

(22) PCT Filed: Aug. 18, 2022
(86) PCT No.: PCT/EP2022/073097

§ 371 Date: Feb. 14, 2024

(30) Foreign Application Priority Data
Aug. 20, 2021 (EP) 21192334.7

(51) Int. Cl. A61K 31/519; A61P 35/02; C07D 487/04

(52) U.S. Cl. CPC A61K 31/519; A61P 35/02; C07D 487/04

(57) ABSTRACT

The present invention refers to quinazoline-thiohydantoin fused heterocycles having the formula (I). The compounds are suitable for treating, ameliorating or preventing a proliferative disorder, such as leukemia, and are useful for treating or ameliorating a multidrug resistant proliferative disorder, such as multidrug resistant leukemia.

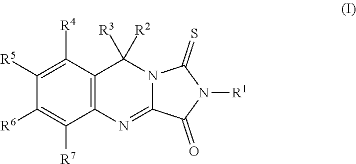

### FIELD OF THE INVENTION

The present invention refers to quinazoline-thiohydantoin fused heterocycles having the formula (I)

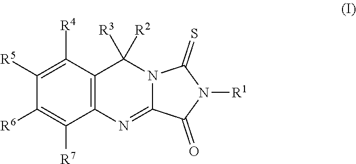

The compounds are suitable for treating, ameliorating or preventing a proliferative disorder, such as leukemia, and can be useful for treating or ameliorating a multidrug resistant proliferative disorder, such as multidrug resistant leukemia.

### BACKGROUND OF THE INVENTION

Most known approved pharmaceuticals contain heterocyclic rings and a great deal of research in heterocyclic chemistry is concerned with the development of new frameworks and of new convenient and efficient synthetic methods for their formation (NPL-1 to NPL-3). Among known heterocycles, quinazolines are known structural fragments in medicinal chemistry (NPL-4 to NPL-8). They possess a wide spectrum of biological properties and are subunits of a variety of natural products and synthetic pharmaceuticals with antiviral, antimalarial, anticancer, and anti-inflammatory activities (NPL-4 to NPL-7).

Functionalization of quinazolines by ring fusion has recently been investigated in drug discovery and development (NPL-13). Quinazolines fused with other heterocyclic units form the frameworks of a variety of pharmacologically important molecules and synthetically accessible marketed drugs like Anagrelide, Evodiamine and Quazinone (NPL-14).

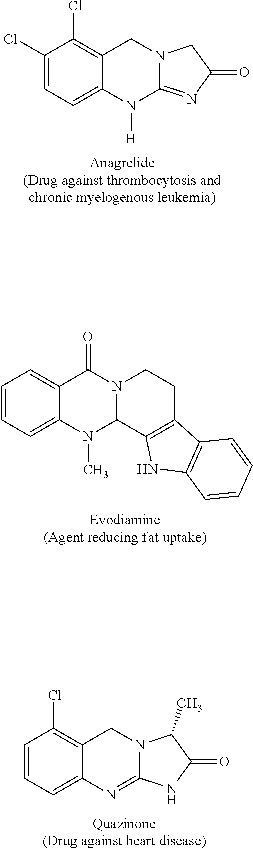 [SMILES: O=C1CN2Cc3c(ccc(Cl)c3Cl)NC2=N1 ; CN1c2ccccc2C(=O)N2CCc3c([nH]c4ccccc34)C21 ; C[C@@H]1C(=O)NC2=Nc3cccc(Cl)c3CN21]

Thiohydantoin is another heterocyclic scaffold (NPL-15). Drugs employing this structural motif include antituberculosis agents (NPL-16), hypolipidemic drugs (NPL-17 and NPL-18), antiviral agents against herpes simplex virus (HSV; NPL-19), antimutagenics (NPL-20), anti-angiogenics (NPL-21), inhibitors of cell division cycle 7 (Cdc7) kinase (NPL-22), inhibitors of fibroblast growth factor receptor 1 (FGFR1) kinase (NPL-23) and drugs against prostate cancer (NPL-24 and NPL-25). While evidently, fusion of quinazolines with different other heterocycles via a nitrogen-bridgehead leads to a variety of highly efficient bioactive quinazoline derivatives, to our knowledge, no examples of fused quinazoline-thiohydantoin frameworks are known yet, since no synthetic approaches for their preparation are available.

The worldwide numbers of leukemia cases, which were published by the American Cancer Society for 2018 are sobering: 437033 new cases and 309006 deaths caused by leukemia (NPL-26). Chemotherapy is still the most promising option for cancer treatment. However, 90% of failure in chemotherapy result from metastasis of cancers due to drug resistance (NPL-27 to NPL-32). Multidrug resistance (MDR), which is the ability of drug resistant tumors to exhibit simultaneous resistance to a number of structurally and functionally unrelated chemotherapeutic agents (NPL-33), is considered a crucial obstacle for an effective clinical cancer chemotherapy (NPL-34). A substantial challenge worldwide is emergent drug resistance in leukemia cells against approved drugs, such as doxorubicin (NPL-35). To address this fundamental issue, research on new anti-leukemia agents is urgently needed (NPL-36).

Therefore, it was an object of the present application to provide compounds which are suitable for treating, ameliorating or preventing a proliferative disorder and more particularly drug resistant proliferative disorder. It was a further object of the present invention to provide compounds which are suitable for treating, ameliorating or preventing leukemia and more particularly drug resistant leukemia. It was a further object of the present invention to provide a simple and efficient method of preparing the respective compounds.

### Non-Patent Literature

NPL-1. Atkins, J. H. & Gershell, L. J. Selective anticancer drugs. Nature Reviews Cancer (2002)
NPL-2. Montalban-Bravo, G. & Garcia-Manero, G. Novel drugs for older patients with acute myeloid leukemia. Leukemia 29, 760-769, doi:10.1038/leu.2014.244 (2015)
NPL-3. Kalaria, P. N., Karad, S. C. & Raval, D. K. A review on diverse heterocyclic compounds as the privileged scaffolds in antimalarial drug discovery. Eur. J. Med. Chem. 158, 917-936, doi:10.1016/j.ejmech.2018.08.040 (2018)
NPL-4. Hutterer, C. et al. The chemical class of quinazoline compounds provides a core structure for the design of anticytomegaloviral kinase inhibitors. Antiviral Res 134, 130-143, doi:10.1016/j.antiviral.2016.08.005 (2016)
NPL-5. Madapa, S. et al. Search for new pharmacophores for antimalarial activity. Part II: synthesis and antimalarial activity of new 6-ureido-4-anilinoquinazolines. Bioorg Med Chem 17, 222-234, doi:10.1016/j.bmc.2008.11.005 (2009)
NPL-6. Zhang, Y. et al. Synthesis and anticancer activities of 5,6,7-trimethoxy-N-phenyl(ethyl)-4-aminoquinazoline derivatives. Eur J Med Chem 66, 335-344, doi:10.1016/j.ejmech.2013.05.043 (2013)
NPL-7. Alafeefy, A. M., Kadi, A. A., Al-Deeb, O. A., El-Tahir, K. E. & Al-Jaber, N. A. Synthesis, analgesic and anti-inflammatory evaluation of some novel quinazoline derivatives. Eur J Med Chem 45, 4947-4952, doi:10.1016/j.ejmech.2010.07.067 (2010)
NPL-8. Held, F. E. et al. Facile access to potent antiviral quinazoline heterocycles with fluorescence properties via merging metal-free domino reactions. Nat. Commun. 8, 15071, doi:10.1038/ncomms15071 (2017)
NPL-9. Saab, A. M. et al. Phytochemical analysis and cytotoxicity towards multidrug-resistant leukemia cells of essential oils derived from Lebanese medicinal plants. Planta Med. 78, 1927-1931 (2012)
NPL-10. Reiter, C. et al. New efficient artemisinin derived agents against human leukemia cells, human cytomegalovirus and Plasmodium falciparum: 2nd generation 1,2,4-trioxane-ferrocene hybrids. Eur. J. Med. Chem. 97, 164-172, doi:http://dx.doi.org/10.1016/j.ejmech.2015.04.053 (2015)
NPL-11. Lubgan, D., Jozwiak, Z., Grabenbauer, G. G. & Distel, L. V. Doxorubicin-transferrin conjugate selectively overcomes multidrug resistance in leukaemia cells. Cell Mol Biol Lett 14, 113-127, doi:10.2478/s11658-008-0037-2 (2009)
NPL-12. Chen, H. Inhibition of human cancer cell line growth and human umbilical vein endothelial cell angiogenesis by artemisinin derivatives in vitro. Pharmacological Research 48, 231-236, doi:10.1016/s1043-6618(03)00107-5 (2003)
NPL-13. Hashem, H. E. Synthesis of Quinazoline and Quinazolinone Derivatives, p. 229. In Quinazolinone and Quinazoline Derivatives. Edited by: Ali Gamal Al-kaf. ISBN 978-1-83880-139-7, eISBN 978-1-83880-140-3; DOI: 10.5772/intechopen.89180 (IntechOpen, 2020)
NPL-14. Panneer Selvam, T., Vijayaraj Kumar, P., Quinazoline Marketed drugs—A Review. Research in Pharmacy 1(1): 1-21 (2011)
NPL-15. Tomasic, T. & Masic, L. P. Rhodanine as a privileged scaffold in drug discovery. Curr Med Chem 16, 1596-1629, doi:10.2174/092986709788186200 (2009)
NPL-16. Archer, S., Unser, M. J. & Froelich, E. Some 5-(Oxoalkyl)-2-thiohydantoins and Their Derivatives. Journal of the American Chemical Society 78, 6182-6185, doi:10.1021/ja01604a064 (1956)
NPL-17. Elwood, J. C., Richert, D. A. & Westerfeld, W. W. A comparison of hypolipidemic drugs in the prevention of an orotic acid fatty liver. Biochemical Pharmacology 21, 1127-1134, doi:10.1016/0006-2952(72)90106-2 (1972)
NPL-18. Tompkins, J. E. 5,5-Diaryl-2-thiohydantoins and 5,5-diaryl-N3-substituted-2-thiohydantoins as potential hypolipidemic agents. J Med Chem 29, 855-859, doi:10.1021/jm00155a042 (1986)
NPL-19. el-Barbary, A. A., Khodair, A. I., Pedersen, E. B. & Nielsen, C. S-glucosylated hydantoins as new antiviral agents. J Med Chem 37, 73-77, doi:10.1021/jm00027a009 (1994)
NPL-20. Takahashi, A., Matsuoka, H., Ozawa, Y. & Uda, Y. Antimutagenic Properties of 3,5-Disubstituted 2-Thiohydantoins. Journal of Agricultural and Food Chemistry 46, 5037-5042, doi:10.1021/jf980430x (1998)
NPL-21. Liu, Y. et al. Anti-angiogenic action of 5,5-diphenyl-2-thiohydantoin-N10 (DPTH-N₁₀). Cancer Lett 271, 294-305, doi:10.1016/j.canlet.2008.06.016 (2008)
NPL-22. Ermoli, A. et al. Cell division cycle 7 kinase inhibitors: 1H-pyrrolo [2,3-b]pyridines, synthesis and structure-activity relationships. J Med Chem 52, 4380-4390, doi:10.1021/jm900248g (2009)
NPL-23. Ravindranathan, K. P. et al. Discovery of novel fibroblast growth factor receptor 1 kinase inhibitors by structure-based virtual screening. J Med Chem 53, 1662-1672, doi:10.1021/jm901386e (2010)
NPL-24. Jung, M. E. et al. Structure-activity relationship for thiohydantoin androgen receptor antagonists for castration-resistant prostate cancer (CRPC). J Med Chem 53, 2779-2796, doi:10.1021/jm901488g (2010)
NPL-25. Yoshino, H. et al. Design and synthesis of an androgen receptor pure antagonist (CH5137291) for the treatment of castration-resistant prostate cancer. Bioorg Med Chem 18, 8150-8157, doi:10.1016/j.bmc.2010.10.023 (2010)
NPL-26. Bray, F. et al. Global cancer statistics 2018: GLOBOCAN estimates of incidence and mortality worldwide for 36 cancers in 185 countries. CA Cancer J Clin 68, 394-424, doi:10.3322/caac.21492 (2018)
NPL-27. Mansoori, B., Mohammadi, A., Davudian, S., Shirjang, S. & Baradaran, B. The different mechanisms of cancer drug resistance: a brief review. Advanced pharmaceutical bulletin 7, 339 (2017)
NPL-28. Goodman, L. S. et al. Nitrogen mustard therapy: Use of methyl-bis (beta-chloroethyl) amine hydrochloride and tris (beta-chloroethyl) amine hydrochloride for hodgkin's disease, lymphosarcoma, leukemia and certain allied and miscellaneous disorders. Journal of the American Medical Association 132, 126-132 (1946)
NPL-29. Steinbach, D. et al. ABCA3 as a possible cause of drug resistance in childhood acute myeloid leukemia. Clin Cancer Res 12, 4357-4363, doi:10.1158/1078-0432.CCR-05-2587 (2006)
NPL-30. Creutzig, U. et al. Idarubicin improves blast cell clearance during induction therapy in children with AML: results of study AML-BFM 93. AML-BFM Study Group. Leukemia 15, 348-354, doi:10.1038/sj.leu.2402046 (2001)
NPL-31. Chessells, J. et al. Clinical features, cytogenetics and outcome in acute lymphoblastic and myeloid leukaemia of infancy: report from the MRC Childhood Leukaemia working party. Leukemia 16, 776 (2002)
NPL-32. Ravindranath, Y. Recent advances in pediatric acute lymphoblastic and myeloid leukemia. Current opinion in oncology 15, 23-35 (2003)
NPL-33. Abdallah, H. M., Al-Abd, A. M., El-Dine, R. S. & El-Halawany, A. M. P-glycoprotein inhibitors of natural origin as potential tumor chemo-sensitizers: A review. Journal of advanced research 6, 45-62 (2015)
NPL-34. Johnstone, R. W., Ruefli, A. A., Tainton, K. M. & Smyth, M. J. A role for P-glycoprotein in regulating cell death. Leukemia & lymphoma 38, 1-11 (2000)
NPL-35. Ramu, A., Pollard, H. B. & Rosario, L. M. Doxorubicin resistance in P388 leukemia—evidence for reduced drug influx. Int J Cancer 44, 539-547, doi:10.1002/ijc.2910440328 (1989)
NPL-36. Szakács, G., Paterson, J. K., Ludwig, J. A., Booth-Genthe, C. & Gottesman, M. M. Targeting multidrug resistance in cancer. Nature reviews Drug discovery 5, 219 (2006)
NPL-37. Ohrnberger, S. et al. Dysregulated serum response factor triggers formation of hepatocellular carcinoma. Hepatology 61, 979-989 (2015)
NPL-38. Hampl, V. et al. Depletion of the transcriptional coactivators megakaryoblastic leukaemia 1 and 2 abolishes hepatocellular carcinoma xenograft growth by inducing oncogene-induced senescence. EMBO Mol. Med. 5, 1367-1382, doi:10.1002/emmm.201202406 (2013)
NPL-39. Voringer, S. et al. Inhibition of TRPM7 blocks MRTF/SRF-dependent transcriptional and tumorigenic activity. Oncogene 39, 2328-2344, doi:10.1038/s41388-019-1140-8 (2020)
NPL-40. Muehlich, S. et al. The transcriptional coactivators megakaryoblastic leukemia ½ mediate the effects of loss of the tumor suppressor deleted in liver cancer 1. Oncogene 31, 3913-3923, doi:10.1038/onc.2011.560 (2012)
NPL-41. Whitson, R. J. et al. Noncanonical hedgehog pathway activation through SRF-MKL1 promotes drug resistance in basal cell carcinomas. Nat. Med. 24, 271-281, doi:10.1038/nm.4476 (2018)

### SUMMARY OF THE INVENTION

The present inventors have found that a compound having the formula (I)

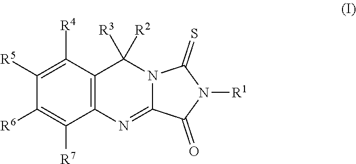

are suitable for treating, ameliorating or preventing a proliferative disorder, such as leukemia, and can be useful for treating or ameliorating a multidrug resistant proliferative disorder, such as multidrug resistant leukemia.

### FIGURES

FIG. 1: Proposed mechanism of the one-pot reaction towards quinazoline-thiohydantoin fused heterocycles catalyzed by a Brønsted acid catalyst (BA catalyst): A 3-step domino reaction with subsequent dehydrogenation.

FIG. 2: Substrate scope of the one-pot synthesis of quinazoline-thiohydantoin fused heterocycles, applying thioureas with achiral residues. A wide variety of achiral thioureas (1a-1s) were used with ethyl glyoxylate 2 to form the desired quinazoline-thiohydantoin heterocycles 3a-3s. The yields are indicated below each entry (3a-3s). The three-step domino procedure was used in the first step with respective thiourea (1.0 equiv.) in DCM (dichloromethane) (Cₜₕᵢₒᵤᵣₑₐ=32.5 mL/mmol), ethyl glyoxylate solution (50% in toluene) (1.5 equiv.) and TFA (trifluoroacetic acid) (0.1 equiv.) at 40° C. for 2 h. In the second step, DDQ (1.1 equiv.) was added and stirred for 1 h at room temperature.

FIG. 3: Substrate scope of the one-pot synthesis of quinazoline-thiohydantoin fused heterocycles, applying thioureas with chiral residues. A wide variety of chiral thioureas (4a-4w) were used with ethyl glyoxylate 2 to form the desired quinazoline-thiohydantoin heterocycles 5a-5w. The yields are indicated below each entry (5a-5w). The standard one-pot procedure was used in the first step with the respective thiourea (1.0 equiv.) in DCM (Cₜₕᵢₒᵤᵣₑₐ=32.5 mL/mmol), ethyl glyoxylate solution (50% in toluene) (1.5 equiv.) and TFA (0.1 equiv.) at 40° C. for 2 h). In the second step, DDQ (1.1 equiv.) was added and stirred for 1 h at room temperature.

FIG. 4: Effect of quinazoline-thiohydantoin fused heterocycles 5g and 5k on MRTF-A nuclear localization and cellular senescence in human leukemia cell line (HAP1).

FIG. 5: (A) One dose cell viability screening;
(B) IC₅₀ values. Five dose (0.01, 0.1, 1, 10, 100 μM) screening.

### DEFINITIONS

Unless defined otherwise, within the meaning of the present application the following definitions apply:

The term “alkyl” refers to a saturated, straight or branched carbon chain which preferably has 1 to 20 carbon atoms, more preferably 1 to 10 carbon atoms, even more preferably 1 to 6carbon atoms, yet more preferably 1 to 4 carbon atoms.

The term “heteroalkyl” refers to an alkyl group in which at least one of the carbon atoms has been replaced by a heteroatom selected from N, O and S.

The term “alkylene” refers to a divalent saturated, straight or branched carbon chain which preferably has 1 to 20 carbon atoms, more preferably 1 to 10 carbon atoms, even more preferably 1 to 6 carbon atoms, yet more preferably 1 to 4 carbon atoms.

The term “alkenyl” refers to a straight or branched carbon chain which contains at least one C═C bond. The C═C bond may be at an end of the carbon chain or within the carbon chain.

The alkenyl preferably has 2 to 20 carbon atoms, more preferably 2 to 10 carbon atoms, even more preferably 2 to 6 carbon atoms, yet more preferably 2 to 4 carbon atoms.

The term “heteroalkenyl” refers to an alkenyl group in which at least one of the carbon atoms has been replaced by a heteroatom selected from N, O and S.

The term “alkinyl” refers to a straight or branched carbon chain which contains at least one C═C bond. The C═C bond may be at an end of the carbon chain or within the carbon chain. The alkinyl preferably has 2 to 20 carbon atoms, more preferably 2 to 10 carbon atoms, even more preferably 2 to 6 carbon atoms, yet more preferably 2 to 4 carbon atoms.

The term “heteroalkenyl” refers to an alkinyl group in which at least one of the carbon atoms has been replaced by a heteroatom selected from N, O and S.

The term “cycloalkyl” represents a cyclic version of “alkyl”. The term “cycloalkyl” is also meant to include bicyclic, tricyclic and polycyclic versions thereof. Unless specified otherwise, the cycloalkyl group can have 3 to 30 carbon atoms, preferably 3 to 20 carbon atoms, more preferably 3 to 10 carbon atoms, even more preferably 5 or 6 carbon atoms.

The term “cycloalkenyl” represents a cyclic version of “alkenyl”. The term “cycloalkenyl” is also meant to include bicyclic, tricyclic and polycyclic versions thereof. Unless specified otherwise, the cycloalkenyl group can have 3 to 30 carbon atoms, preferably 3 to 20 carbon atoms, more preferably 3 to 10 carbon atoms, even more preferably 5 or 6 carbon atoms.

“Hal” or “halogen” represents F, Cl, Br and I.

The term “carbocyclic moiety” refers to a saturated, unsaturated or aromatic ring system which can contain 3 to 30 carbon ring atoms, preferably 3 to 20 carbon ring atoms, more preferably 3 to 10 carbon ring atoms, even more preferably 5 to 10 carbon ring atoms. The term “carbocyclic moiety” is also meant to include bicyclic, tricyclic and polycyclic versions thereof. The carbocyclic ring system can contain one or more rings (such as 1 to 5 rings) which can be fused, spirocyclic or bridged cyclic compounds. The “carbocyclic moiety” can contain one or more cycloalkyl, cycloalkenyl, and/or aryl rings.

The term “heterocyclic moiety” refers to a saturated, unsaturated or heteroaromatic ring system which can contain 3 to 30 ring atoms, preferably 3 to 20 ring atoms, more preferably 3to 10 ring atoms, even more preferably 5 to 10 carbon atoms. The term “heterocyclic moiety” is also meant to include bicyclic, tricyclic and polycyclic versions thereof. The heterocyclic ring system can contain one or more rings (such as 1 to 5 rings) which can be fused, spirocyclic or bridged cyclic compounds. The “heterocyclic moiety” can contain one or more cycloheteroalkyl, cycloheteroalkenyl, and/or heteroaryl rings. The heterocyclic moiety can contain one or more three-, four-, five-, six-or seven-membered rings, wherein one or more of the carbon atoms in the ring have been replaced by 1 or 2 (for the three-membered ring), 1, 2 or 3 (for the four-membered ring), 1, 2, 3, or 4 (for the five-membered ring) or 1, 2, 3, 4, or 5 (for the six-membered ring) and 1, 2, 3, 4, 5 or 6 (for the seven-membered ring) of the same or different heteroatoms, wherein the heteroatoms are selected from O, N and S. If the heterocyclic moiety contains more than one ring, the rings can be heterocyclic or carbocyclic with at least one ring being heterocyclic.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphthyl or anthracenyl, preferably phenyl.

The term “heteroaryl” preferably refers to a five-or six-membered aromatic ring wherein one or more of the carbon atoms in the ring have been replaced by 1, 2, 3, or 4 (for the five-membered ring) or 1, 2, 3, 4, or 5 (for the six-membered ring) of the same or different heteroatoms, whereby the heteroatoms are selected from O, N and S. Examples of the heteroaryl group include pyrrole, pyrrolidine, oxolane, furan, imidazolidine, imidazole, triazole, tetrazole, pyrazole, oxazolidine, oxazole, thiazole, piperidine, pyridine, morpholine, piperazine, and dioxolane.

If a compound or moiety is referred to as being “optionally substituted”, it can in each instance include 1 or more of the indicated substituents, whereby the substituents can be the same or different.

The term “pharmaceutically acceptable salt” refers to a salt of a compound of the present invention. Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counter anions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

Compounds of the invention may have one or more optically active carbons can exist as racemates and racemic mixtures, stereoisomers (including diastereomeric mixtures and individual diastereomers, enantiomeric mixtures and single enantiomers, mixtures of conformers and single conformers), tautomers, atropisomers, and rotamers. All isomeric forms are included in the present invention. Compounds described in this specification containing olefinic double bonds include E and Z geometric isomers. Also included in this invention are all salt forms, polymorphs, hydrates and solvates.

The term “polymorphs” refers to the various crystalline structures of the compounds of the invention. This may include, but is not limited to, crystal morphologies (and amorphous materials) and all crystal lattice forms. Salts can be crystalline and may exist as more than one polymorph.

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds of formula (II) can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18th ed., Mack Publishing Company, Easton, PA, 1990, p. 1445, the disclosure of which is hereby incorporated by reference.

The compound of the invention can also be provided in the form of a prodrug, namely a compound which is metabolized in vivo to the active metabolite.

### DETAILED DESCRIPTION OF THE INVENTION

The invention is summarized by the embodiments listed in the claims. It is understood that combinations of all of the preferred embodiments listed hereinafter and in the claims are contemplated as being within the scope of the present invention.

The present invention relates to a compound having the formula (I)

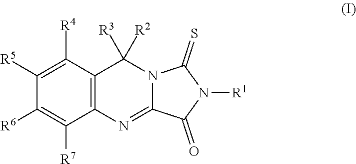

R¹ is selected from alkyl, alkenyl, alkinyl, a carbocyclic moiety, a heterocyclic moiety, an -L-carbocyclic moiety, and an -L-heterocyclic moiety, wherein the alkyl, alkenyl, and alkinyl can optionally contain one or more catenary oxygen, nitrogen or sulfur atoms.

In a first embodiment, R¹ is selected from a carbocyclic moiety, a heterocyclic moiety, an -L-carbocyclic moiety, and an -L-heterocyclic moiety, preferably R¹ is selected from an -L-carbocyclic moiety, and an -L-heterocyclic moiety. In this embodiment, the carbocyclic moiety and the heterocyclic moiety are selected from a pharmacophore. Within the meaning of the present application a pharmacophore refers to a residue of a pharmaceutically active agent, wherein the pharmaceutically active agent has the structure R*—O-pharmacophore, with R* being H or C₁₋₄ alkyl. The pharmaceutically active agent is not particularly limited with the exception that it has an —O—R* moiety. Examples thereof include agents against proliferative disorders (preferably against leukemia, breast cancer, skin cancer, lung cancer, human embryonic kidney cells, or cervical cancer, more preferably against leukemia), anti-inflammatory agents, antiviral agents. Specific pharmaceutically active agents include anti-inflammatory and anti-cancer agents e.g., aspirin, betulin, 23-hydroxybetulin, egonol, thymoquinone, 5-fluorouracil (5-FU), erlotinib, gefitinib, vandetanib, epothilone, daunorubicin, doxorubicin, paclitaxel, vinblastine, docetaxel, mitoxantrone, cytarabine, trabectedin, eribulin, minocycline, raloxifene, tamoxifene, mitomycin C, ciprofloxacin, camptothecin, tobramycin, curcumin, gemcitabine, cisplatin, and melphalan.

In a second embodiment, R¹ is selected from alkyl, a carbocyclic moiety, a heterocyclic moiety, an -L-carbocyclic moiety, and an -L-heterocyclic moiety, wherein the alkyl can optionally contain one or more catenary oxygen, nitrogen or sulfur atoms.

In a third embodiment, R¹ is selected from alkyl, wherein the alkyl can optionally contain one or more catenary oxygen, nitrogen or sulfur atoms, preferably the alkyl has the formula —C(H)(CH₃)-alkyl, wherein the alkyl can optionally contain one or more catenary oxygen, nitrogen or sulfur atoms.

In any of the above embodiments, the alkyl preferably does not contain one or more catenary oxygen, nitrogen or sulfur atoms.

In any of the above embodiments, the carbocyclic moiety if present is preferably substituted.

In any of the above embodiments, the heterocyclic moiety if present is preferably substituted.

In fourth embodiment, R¹ is selected from a carbocyclic moiety, a heterocyclic moiety, an -L-carbocyclic moiety, and an -L-heterocyclic moiety, preferably an -L-carbocyclic moiety, and an -L-heterocyclic moiety, more preferably -L-aryl or -L-heteroaryl, even more preferably -L-aryl.

In the second to fourth embodiments of R¹, the carbocyclic moiety is as defined in the “Definitions” section. Preferably the carbocyclic moiety can be selected from cycloalkyl and aryl, more preferably aryl. Specific examples thereof include cyclopentyl, cyclohexyl, phenyl, naphthyl and cyclohexyl which is fused to phenyl, more preferably phenyl.

In the second to fourth embodiments of R¹, the heterocyclic moiety is as defined in the “Definitions” section. Preferably the heterocyclic moiety can be selected from heterocycloalkyl and heteroaryl, more preferably heteroaryl. Specific examples thereof include pyridine, quinoline, furan and thiophene.

In a preferred embodiment, R¹ is —C(H)(CH₃)-aryl, such as —C(H)(CH₃)-phenyl.

Examples of the catenary oxygen, nitrogen or sulfur atoms include —O—, —S— and —N(R**)—, with R** being selected from H and C₁₋₄ alkyl.

R² is selected from H, alkyl, alkenyl, alkinyl, a carbocyclic moiety, a heterocyclic moiety, an -L-carbocyclic moiety, and an -L-heterocyclic moiety; preferably R² is selected from H, alkyl, a carbocyclic moiety and a heterocyclic moiety; more preferably R² is selected from H and alkyl; even more preferably R² is H.

R³ is selected from H, alkyl, alkenyl, alkinyl, a carbocyclic moiety, a heterocyclic moiety, an -L-carbocyclic moiety, and an -L-heterocyclic moiety; preferably R³ is selected from H, alkyl, a carbocyclic moiety and a heterocyclic moiety; more preferably R³ is selected from H and alkyl; even more preferably R³ is H.

R⁴ is selected from H, alkyl, alkenyl, alkinyl, a carbocyclic moiety, a heterocyclic moiety, an -L-carbocyclic moiety, an -L-heterocyclic moiety, halogen, —NO₂, —O—R⁸, —S—R⁸, —CN, —OCN, —SCN, —NCS, —N₃ and —NR^{a}R^{b}; preferably R⁴ is selected from H, alkyl, halogen, —NO₂, —O—R⁸, —S—R⁸, —CN, —OCN, —SCN, —NCS, —N₃ and —NR^{a}R^{b}; more preferably R⁴ is selected from H, alkyl, halogen, —NO₂, and —O—R⁸; even more preferably H, halogen, NO₂, —OH, and —O-alkyl.

R⁵ is selected from H, alkyl, alkenyl, alkinyl, a carbocyclic moiety, a heterocyclic moiety, an -L-carbocyclic moiety, an -L-heterocyclic moiety, halogen, —NO₂, —O—R⁸, —S—R⁸, —CN, —OCN, —SCN, —NCS, —N₃ and —NR^{a}R^{b}; preferably R⁵ is selected from H, alkyl, halogen, —NO₂, —O—R⁸, —S—R⁸, —CN, —OCN, —SCN, —NCS, —N₃ and —NR^{a}R^{b}; more preferably R⁵ is selected from H, alkyl, halogen, —NO₂, and —O—R⁸; even more preferably H, halogen, NO₂, —OH, and —O-alkyl.

R⁶ is selected from H, alkyl, alkenyl, alkinyl, a carbocyclic moiety, a heterocyclic moiety, an -L-carbocyclic moiety, an -L-heterocyclic moiety, halogen, —NO₂, —O—R⁸, —S—R⁸, —CN, —OCN, —SCN, —NCS, —N₃ and —NR^{a}R^{b}; preferably R⁶ is selected from H, alkyl, halogen, —NO₂, —O—R⁸, —S—R⁸, —CN, —OCN, —SCN, —NCS, —N₃ and —NR^{a}R^{b}; more preferably R⁶ is selected from H, alkyl, halogen, —NO₂, and —O—R⁸; even more preferably H, halogen, NO₂, —OH, and —O-alkyl.

R⁷ is selected from H, alkyl, alkenyl, alkinyl, a carbocyclic moiety, a heterocyclic moiety, an -L-carbocyclic moiety, an -L-heterocyclic moiety, halogen, —NO₂, —O—R⁸, —S—R⁸, —CN, —OCN, —SCN, —NCS, —N₃ and —NR^{a}R^{b}; preferably R⁷ is selected from H, alkyl, halogen, —NO₂, —O—R⁸, —S—R⁸, —CN, —OCN, —SCN, —NCS, —N₃ and —NR^{a}R^{b}; more preferably R⁷ is selected from H, alkyl, halogen, —NO₂, and —O—R⁸; even more preferably H, halogen, NO₂, —OH, and —O-alkyl.

R⁸ is independently selected from H, alkyl, alkenyl, alkinyl, a carbocyclic moiety, and a heterocyclic moiety; preferably R⁸ is independently selected from H, alkyl, a carbocyclic moiety, and a heterocyclic moiety; more preferably R⁸ is independently selected from H and alkyl; even more preferably R⁸ is H.

R^{a} is selected from H, alkyl, a carbocyclic moiety, and a heterocyclic moiety; preferably R^{a} is selected from H, and alkyl; more preferably R^{a} is H.

R^{b} is selected from H, alkyl, a carbocyclic moiety, and a heterocyclic moiety; preferably R^{b} is selected from H, and alkyl; more preferably R^{b} is H.

L is independently selected from an alkylene moiety, the chain of which can be optionally interrupted by one or more —O—, —S—, —N(R)—, —C(O)—, —C(O)—O—, —N(R)—C(O)—O—, and —C(O)—N(R)—; preferably L is independently selected from an alkylene moiety, the chain of which can be optionally interrupted by one or more —O—, —S—, —N(R)—, —C(O)—O—, —N(R)—C(O)—O—, and —C(O)—N(R)—; more preferably L is independently selected from an alkylene moiety, the chain of which can be optionally interrupted by one or more —O—. In one embodiment, L is selected from —C(H)(CH₃)—.

R is independently selected from H and alkyl; preferably R is H.

In one option, R¹ is selected from an -L-carbocyclic moiety and an -L-heterocyclic moiety, and

L is independently selected from an alkylene moiety, the chain of which can be optionally interrupted by one or more —O—.

In a further option, R¹ is selected from an -L-aryl, and

L is independently selected from an alkylene moiety, the chain of which can be optionally interrupted by one or more —O—.

In yet another option, R⁴, R⁵, R⁶, and R⁷ are independently selected from H, halogen, NO₂, —OH, and —O-alkyl, in particular wherein one or two of R⁴, R⁵, R⁶, and R⁷ is independently selected from halogen, —NO₂, —OH, and —O-alkyl and the others of R⁴, R⁵, R⁶, and R⁷ are H.

In a further option, R² and R³ are both H.

In the above definitions, the alkyl, alkenyl, alkinyl, alkylene, carbocyclic moiety and heterocyclic moiety can be optionally substituted one or more times.

The optional substituents of the alkyl, alkenyl, alkinyl, and alkylene are not particularly limited and can be selected from a carbocyclic moiety, a heterocyclic moiety, -halogen, —NO₂, —O—R#, —S—R#, —CN, —OCN, —SCN, —NCS, —C(O)—R#, —C(O)—O—R#, —N(R#)—C(O)—O—R#, and —C(O)—N(R#)—R#, —N₃ and —NR#R#, with R# being independently selected from H and alkyl.

The optional substituents of the alkyl, alkenyl, and alkinyl of R² to R⁸, R^{a}, R^{b} and R can be preferably selected from-halogen, and —O—R#. More preferably the alkyl, alkenyl, and alkinyl of R² to R⁸, R^{a}, R^{b} and R are unsubstituted.

The optional substituents of the alkyl, alkenyl, and alkinyl of R¹ are not particularly limited and are preferably selected from-halogen, —NO₂, —O—R#, —S—R#, —CN, —OCN, —SCN, —NCS, —C(O)—R#, —C(O)—O—R#, —N(R#)—C(O)—O—R#, and —C(O)—N(R#)—R#, —N₃ and —NR#R#, with R# being independently selected from H and alkyl, more preferably the optional substituents are selected from -halogen, —NO₂, —O—R#, —S—R#, —CN, —C(O)—R#, —C(O)—O—R#, —N(R#)—C(O)—O—R#, and —C(O)—N(R#)—R#, and —NR#R#; even more preferably-halogen, —NO₂, —O—R#, —S—R#, —C(O)—O—R#, —N(R#)—C(O)—O—R#, and —C(O)—N(R#)—R#, and —NR#R#. In one embodiment, the alkyl, alkenyl, and alkinyl of R¹ can be unsubstituted.

The optional substituents of the alkylene are preferably selected from-halogen, —NO₂, —O—R#, —S—R#, —CN, —OCN, —SCN, —NCS, —C(O)—R#, —C(O)—O—R#, —N(R#)—C(O)—O—R#, and —C(O)—N(R#)—R#, —N₃ and —NR#R#, with R#being independently selected from H and alkyl. In one embodiment, the alkylene can be unsubstituted.

The optional substituents of the carbocyclic moiety and heterocyclic moiety are not particularly limited and can be selected from alkyl, alkenyl, alkinyl, a carbocyclic moiety, a heterocyclic moiety, -halogen, —NO₂, —O—R#, —S—R#, —CN, —CF₃, —OCN, —SCN, —NCS, —C(O)—R#, —C(O)—O—R#, —N(R#)—C(O)—O—R#, and —C(O)—N(R#)—R#, —N₃ and —NR#R#, with R# being independently selected from H and alkyl. In a preferred embodiment, the optional substituents of the carbocyclic moiety and heterocyclic moiety can be selected from alkyl, a carbocyclic moiety, a heterocyclic moiety, -halogen, —NO₂, —O—R#, —S—R#, —CN, —CF₃, —C(O)—R#, —C(O)—O—R#, —N(R#)—C(O)—O—R#, and —C(O)—N(R#)—R#, and —NR#R#; more preferably alkyl, a carbocyclic moiety, a heterocyclic moiety, -halogen, —NO₂, —O—R#, —CF₃, and —NR#R#.

The optional substituents of the carbocyclic moiety and heterocyclic moiety of R¹ are not particularly limited and can be selected from alkyl, alkenyl, alkinyl, a carbocyclic moiety, a heterocyclic moiety, -halogen, —NO₂, —O—R#, —S—R#, —CN, —OCN, —SCN, —NCS, —C(O)—R#, —C(O)—O—R#, —N(R#)—C(O)—O—R#, and —C(O)—N(R#)—R#, —N₃ and —NR#R#, with R# being independently selected from H and alkyl. In a preferred embodiment, the optional substituents of the carbocyclic moiety and heterocyclic moiety can be selected from alkyl, a carbocyclic moiety, a heterocyclic moiety, -halogen, —NO₂, —O—R#, —S—R#, —CN, —CF₃, —C(O)—R#, —C(O)—O—R#, —N(R#)—C(O)—O—R#, and —C(O)—N(R#)—R#, and —NR#R#; more preferably alkyl, a carbocyclic moiety, a heterocyclic moiety, -halogen, —NO₂, —O—R#, —CF₃, and —NR#R#.

The optional substituents of the carbocyclic moiety and heterocyclic moiety of R² to R⁸, R^{a}, R^{b} and R are not particularly limited and can be selected from alkyl, alkenyl, alkinyl, a carbocyclic moiety, a heterocyclic moiety, -halogen, —NO₂, —O—R#, —S—R#, —CN, —OCN, —SCN, —NCS, —C(O)—R#, —C(O)—O—R#, —N(R#)—C(O)—O—R#, and —C(O)—N(R#)—R#, —N₃ and —NR#R#, with R# being independently selected from H and alkyl. In a preferred embodiment, the optional substituents of the carbocyclic moiety and heterocyclic moiety can be selected from alkyl, a carbocyclic moiety, a heterocyclic moiety, -halogen, —NO₂, —O—R#, —S—R#, —CN, —CF₃, —C(O)—R#, —C(O)—O—R#, —N(R#)—C(O)—O—R#, and —C(O)—N(R#)—R#, and —NR#R#; more preferably alkyl, a carbocyclic moiety, a heterocyclic moiety, -halogen, —NO₂, —O—R#, —CF₃, and —NR#R#; even more preferably alkyl, -halogen, —NO₂, —O—R #, —CF₃, and —NR#R#. In one embodiment, the carbocyclic moiety and heterocyclic moiety of R² to R⁸, R^{a}, R^{b} and R are unsubstituted.

The compounds of the present invention can be administered to a patient in the form of a pharmaceutical composition which can optionally comprise one or more pharmaceutically acceptable excipient(s) and/or carrier(s).

As used herein the term “therapeutically effective amount” refers to an amount sufficient to elicit the desired biological response. In the present invention the desired biological response is the treatment, amelioration or prevention of a proliferative disorder.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations. The skilled person knows that the effective amount of pharmaceutical compositions administered to an individual will depend, inter alia, on the nature of the compound.

The compounds of the present invention can be administered by various well known routes, including oral, rectal, intracistemally, intravaginally, intraperitoneally, topically, bucally, intragastrical, intracranial and parenteral administration, e.g. intravenous, intramuscular, intranasal, intradermal, subcutaneous, and similar administration routes. Oral, and parenteral administration are particularly preferred. Depending on the route of administration different pharmaceutical formulations are required and some of those may require that protective coatings are applied to the drug formulation to prevent degradation of a compound of the invention in, for example, the digestive tract.

Thus, preferably, a compound of the invention is formulated as a syrup, an infusion or injection solution, a spray, a tablet, a capsule, a capslet, lozenge, a liposome, a suppository, a plaster, a band-aid, a retard capsule, a powder, or a slow release formulation.

Particular preferred pharmaceutical forms for the administration of a compound of the invention are forms suitable for injectionable use and include sterile aqueous solutions or dispersions and sterile powders for the extemporaneous preparation of sterile injectable solutions or dispersions. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half-life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride, may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions can be vacuum-dried or freeze-dried as necessary. Preferred diluents of the present invention are water, physiological acceptable buffers, physiological acceptable buffer salt solutions or salt solutions. Preferred carriers are cocoa butter and vitebesole. Excipients which can be used with the various pharmaceutical forms of a compound of the invention can be chosen from the following non-limiting list:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscarmellose, sodium methyl cellulose, agar, bentonite, alginic acid, carboxymethyl cellulose, polyvinyl pyrrolidone and the like.

In one embodiment the formulation is for oral administration and the formulation comprises one or more or all of the following ingredients: pregelatinized starch, talc, povidone K 30,croscarmellose sodium, sodium stearyl fumarate, gelatin, titanium dioxide, sorbitol, monosodium citrate, xanthan gum, titanium dioxide, flavoring, sodium benzoate and saccharin sodium.

Other suitable excipients can be found in the Handbook of Pharmaceutical Excipients, published by the American Pharmaceutical Association, which is herein incorporated by reference.

It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. It is contemplated that the dosage of a compound of the invention in the therapeutic or prophylactic use of the invention should be in the range of about 0.1 mg to about 1 g of the active ingredient (i.e. compound of the invention) per kg body weight. However, in a preferred use of the present invention a compound of the invention is administered to a subject in need thereof in an amount ranging from 1.0 to 500 mg/kg body weight, preferably ranging from 1 to 200 mg/kg body weight. The duration of therapy with a compound of the invention will vary, depending on the severity of the disease being treated and the condition and idiosyncratic response of each individual patient. In one preferred embodiment of a prophylactic or therapeutic use, from 10 mg to 200 mg of the compound are orally administered to an adult per day, depending on the severity of the disease and/or the degree of exposure to disease carriers.

As is known in the art, the pharmaceutically effective amount of a given composition will also depend on the administration route. In general, the required amount will be higher if the administration is through the gastrointestinal tract, e.g., by suppository, rectal, or by an intragastric probe, and lower if the route of administration is parenteral, e.g., intravenous. Typically, a compound of the invention will be administered in ranges of 50 mg to 1 g/kg body weight, preferably 10 mg to 500 mg/kg body weight, if rectal or intragastric administration is used and in ranges of 1 to 100 mg/kg body weight if parenteral administration is used. For intranasal administration, 1 to 100 mg/kg body weight are envisaged.

If a person is known to be at risk of developing a disease treatable with a compound of the invention, prophylactic administration of the compound of the present invention or the pharmaceutical composition according to the invention may be possible.

In one aspect, the present invention relates to the compound of the present invention, a stereoisomer, racemic mixture, tautomer, pharmaceutically acceptable salt, prodrug, hydrate, solvate, or polymorph thereof for use in the treatment, amelioration or prevention of a proliferative disorder.

In a second aspect, the present invention refers to the use of the compound of the present invention, a stereoisomer, racemic mixture, tautomer, pharmaceutically acceptable salt, prodrug, hydrate, solvate, or polymorph thereof for the manufacture of a medicament for treating, ameliorating or preventing a proliferative disorder.

In a third aspect, the present invention is directed to a method of treating, ameliorating or preventing a proliferative disorder, in which a therapeutically effective amount of the compound of the present invention, a stereoisomer, racemic mixture, tautomer, pharmaceutically acceptable salt, prodrug, hydrate, solvate, or polymorph thereof is administered to a patient in need thereof.

The term “proliferative disorder” refers to any disorder which is characterized by an excessive increase in the number of cells. This covers both benign and malign, particularly malign, proliferative disorders. Examples thereof include cancers such as leukemia, breast cancer, skin cancer, lung cancer, human embryonic kidney cells, or cervical cancer, preferably leukemia. It has been surprisingly found that the compounds of the present invention are suitable for treating multidrug resistant proliferative disorders, such as multidrug resistant cancers, including multidrug resistant leukemia, breast cancer, skin cancer, lung cancer, human embryonic kidney cells, or cervical cancer, preferably multidrug resistant leukemia such as P glycoprotein-overexpressing CEM/ADR5000 leukemia cells.

Within the context of the present invention, multidrug resistance (MDR) means the ability of a proliferative disorder to exhibit simultaneous resistance to a number of structurally and functionally unrelated chemotherapeutic agents.

The compounds of the present invention can be prepared by any appropriate techniques. One such method comprises the steps of:
(i) providing a compound having formula (II)

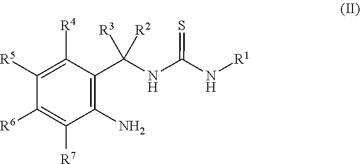

(ii) reacting the compound having the formula (II) with a compound having the formula (III)

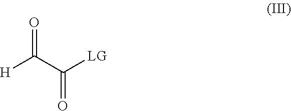

wherein R¹, R², R³, R⁴, R⁵, R⁶, and R⁷ are as defined above; and
LG is a leaving group such as —O-alkyl, —OH, —O-aryl, -halogen, —O—SO₂CF₃, tosylates, or mesylates, preferably LG is —O-alkyl.

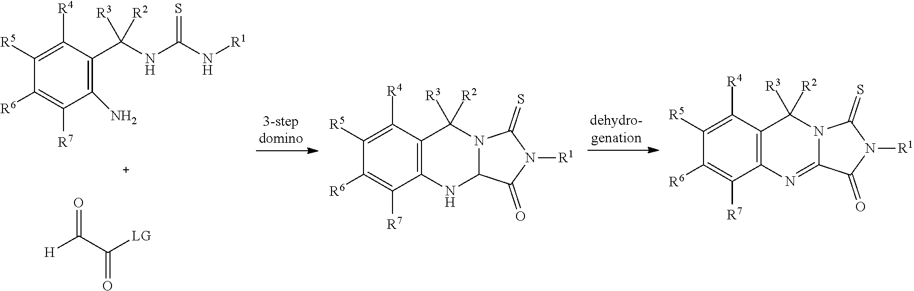

The reaction between the compound having the formula (II) and the compound having the formula (III) can be conducted by a three-step domino reaction followed by a dehydrogenation reaction. This method is a highly efficient, operationally simple and fully metal-free reaction which can be conducted as a four-step one-pot process. Merging a new three-step domino reaction and a dehydrogenation step in one-pot leads to atom-economic, expeditious and high-yielding (up to 92%) organic synthesis using easily available starting materials.

The domino reaction can be conducted by reacting the compound having the formula (II) and the compound having the formula (III).

The amount of the compound having the formula (III) can range from about 0.1 equivalents to about 10 equivalents, preferably about 0.5 equivalents to about 2.5 equivalents, preferably about 1.5 equivalents based on 1 equivalent of the compound having the formula (II).

The domino reaction can be conducted in any suitable solvent, preferably in dichloromethane. Examples of possible solvents for the subsequent dehydrogenation step include halogenated hydrocarbon solvents (such as dichloromethane, chloroform), EtOAc and EtOH as well as mixtures thereof, preferably halogenated hydrocarbon solvents.

The temperature at which the three-step domino reaction is conducted is not particularly limited and can range from about r.t. to about 40° C., more preferably about 40° C. The temperature should not exceed the boiling point of the solvent.

Typical reaction durations range from about 30 minutes to about 48 hours, more typically about 30 minutes to about 24 hours. The reaction duration can be chosen by a skilled person suitably depending on the chosen reaction temperature.

A Brønsted acid, which is preferably selected from trifluoroacetic acid, phosphoric acid diesters, acetic acid, and ArCOOH, more preferably trifluoroacetic acid, can be used to catalyze and accelerate the domino reaction. The amount of the Brønsted acid will depend on the specific compound chosen and can range from about 5 mol % to about 20 mo %.

When the domino reaction has been conducted to completion or to the desired extent, the dehydrogenation reaction can be conducted. If desired, the product of the domino reaction can be isolated and optionally purified before it is subjected to the dehydrogenation reaction. However, the dehydrogenation reaction is preferably conducted in a one-pot manner by employing the reaction mixture of the domino reaction as such without any intermediate work-up.

The dehydrogenation reaction can be conducted in any suitable solvent. Examples of possible solvents include halogenated hydrocarbon solvents (such as dichloromethane, chloroform), ester solvents (such as acetic acid ethyl ester), alcohol solvents (such as methanol, ethanol, isopropanol, butanol) as well as mixtures thereof, preferably halogenated hydrocarbon solvents, ester solvents, and alcohol solvents. Typically the hydrogenation reaction will be conducted in the same solvent as the domino reaction.

The temperature at which the hydrogenation reaction is conducted is not particularly limited and can range from about 10 to about 80° C., more preferably about 20 to about 70° C. The temperature should not exceed the boiling point of the solvent.

Typical reaction durations range from about 30 minutes to about 200 hours, more typically about 30 minutes to about 150 hours. The reaction duration can be chosen by a skilled person suitably depending on the chosen reaction temperature and employed oxidant.

The hydrogenation reaction can be conducted in the presence of an oxidant. The oxidant is not particularly limited and can be selected from air, oxygen, 2,3-dichloro-5,6-dicyano-1,4-benzoquinone (DDQ), a combination of hydrogen peroxide-urea (UHP) and iodine, manganese oxide; preferably DDQ, a combination of UHP and iodine, and manganese oxide; more preferably DDQ. The amount of the oxidant can be chosen by a skilled person depending on the type of oxidant chosen. For DDQ the amount will typically range from about 1.0 equiv.

to about 1.2 equiv. For UHP and iodine the amount will typically range from about 1.5 equiv. to about 2.0 equiv. For manganese oxide the amount will typically range from about 1.0 equiv. to about 1.5 equiv.

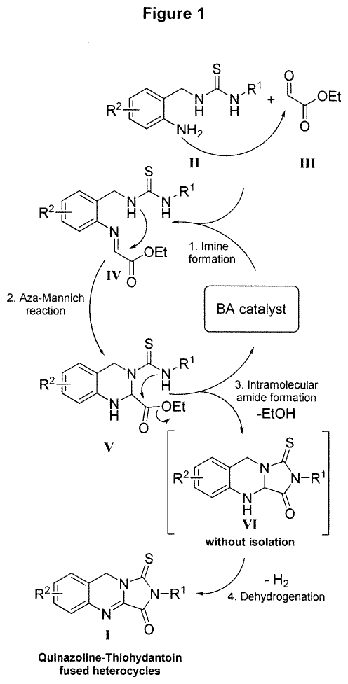

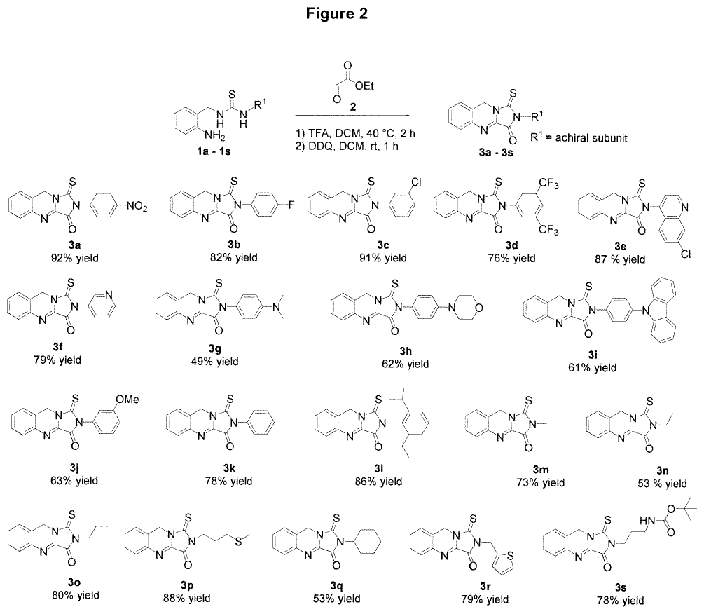

Although not wishing to be bound by theory, the reaction mechanism shown in FIG. 1 is proposed.

In the first step of the three-step domino reaction, the primary amine of the thiourea (II) reacts with the aldehyde function of the compound (III) to form imine (IV). In the second step, the amine of the thiourea moiety of (IV) can attack intramolecular imine (IV) to close a six-membered ring and yield a cyclic aminal (V). To form the thiohydantoin moiety, the secondary amine and the ester function of (V) undergo an intramolecular amide formation and (VI) is released in the third step of the domino reaction from the catalytic cycle. Acid catalysis leads to a significant acceleration of the domino reaction. The desired quinazoline backbone can be obtained via a subsequent dehydrogenation reaction to form the final product (I).

The present invention will be described with respect to the following examples which, however, should not be construed as limiting.

### EXAMPLES

### General Procedure for the Synthesis of Quinazoline-Thiohydantoin Fused Heterocycles

The respective thiourea 1a-1s, 4a-4w (1.0 equiv.) was dissolved in DCM (Cₜₕᵢₒᵤᵣₑₐ=33 mL/mmol), ethyl glyoxylate solution (50% in toluene) (1.5 equiv.) and TFA (0.1 equiv.) were added and the mixture was heated to 40° C. for 2 h. The solution was cooled down to room temperature and DDQ (1.1 equiv.) was added. After stirring for one additional hour at room temperature the solvent was removed under reduced pressure and flash column chromatography (DCM) was performed to obtain the pure quinazoline-thiohydantoin fused heterocycles.

The three-step domino reaction and a dehydrogenation reaction were conducted as a one-pot reaction in DCM. Thus, these two reaction steps were optimized separately.

The reaction conditions of the three-step domino reaction were optimized first (Table 1). As a model system commercially available ethyl glyoxylate 2 (50 mol % in toluene) and 1-(2-aminobenzyl)-3-(4-nitrophenyl)thiourea 1a were used. The thiourea 1a can be obtained easily starting from commercially available substrates on a large scale. The dehydrogenation step was performed in entry 1 and 2 at room temperature within one hour. Dichloromethane was used as a solvent and trifluoroacetic acid (10 mol %) was added to catalyze and accelerate the domino reaction. The reaction velocity significantly accelerated from 20 h (entry 1) to 2 h (entry 2) by increasing the temperature of the reaction from room temperature to 40° C. In addition, the yield raised from 54% (entry 1) to 92% (entry 2).

TABLE 1
Optimization of the dehydrogenation conditions.ª

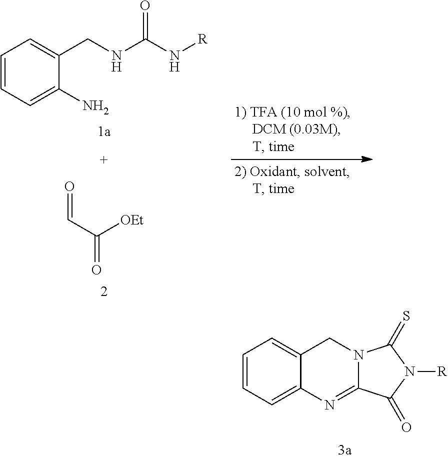

| | domino reaction | | dehydrogenation | | | | |
|---|---|---|---|---|---|---|---|
| | t (h) | T (° C.) | Oxidant | t (h) | T (° C.) | Solvent | Yield^{b} (%) |
| 1 | 20 | r. t. | DDQ | 1 | r. t. | DCM | 54 |
| 2 | 2 | 40 | DDQ | 1 | r. t. | DCM | 92 |
| 3 | 2 | 40 | MnO₂^{c} | 3 | 62 | CHCl₃ | — |
| 4 | 2 | 40 | O₂ (air) | 100 | r. t. | DCM | 74 |
| 5 | 2 | 40 | O₂ | 72 | r. t. | DCM | 87 |
| 6 | 2 | 40 | UHP^{d}, I₂^{e} | 5 | r. t. | EtOAc/EtOH^{f} | 90 |

^{a}All reactions were carried out using 1a (1.0 equiv), 2 (1.5 equiv.), TFA (10 mol %) DCM (0.03M), 40 °C, 2h. Then addition of oxidant under indicated conditions.
^{b}Isolated yield.
^{c}1.5 equiv.
^{d}2.0 equiv.
^{e}0.2 equiv.
^{f}2:1 (v/v).
r.t. = room temperature

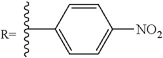 [SMILES: *c1ccc([N+](=O)[O-])cc1]

With these conditions for the three-step domino reaction in hand the dehydrogenation reaction was investigated. Since high temperature in this step (entry 3) led to decomposition of the substrate 1a, an oxidizing agent working under mild conditions was employed. Oxygen from ambient air, as a green and renewable agent, could perform the dehydrogenation at room temperature with a yield of 74% (entry 4) and the yield of the reaction could be even further increased to 87% by using higher oxygen pressure from an oxygen balloon (entry 5). The reaction times could be shortened by applying a combination of hydrogen peroxide-urea (UHP) and iodine to the reaction (entry 6). Very good results were obtained by using 2,3-dichloro-5,6-dicyano-1,4-benzoquinone (DDQ) (shown in entry 2). Mild conditions, a short reaction time of one hour and a yield of 92% made DDQ the optimal reagent.

This reaction sequence was subsequently applied to a variety of thioureas with achiral subunits (1a-1s). Electron donating systems next to the thiourea moiety (R¹) might facilitate the intramolecular amide formation and yields from 49% to 92% were obtained for substrates with aromatic systems next to the thiourea moiety (3a-3l). Groups with a strong electron withdrawing effect on the aromatic system (3b and 3d) slightly lowered the yield. The reaction could also be performed with alkyl moieties bound to the thiourea group (3m-3s) with moderate to very good yields. Probably due to less sterical hindrance, substrates with a secondary carbon (3r) next to the amine reacted faster than one with a tertiary carbon center (3q).

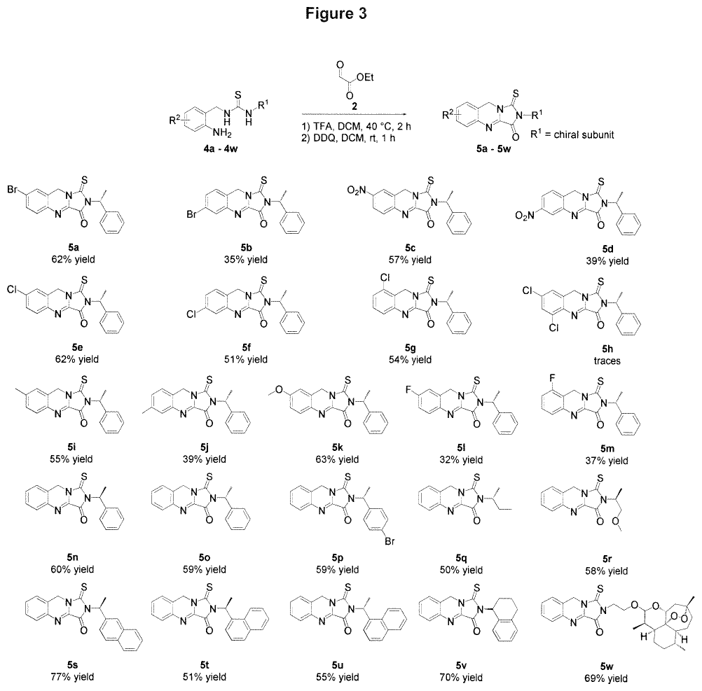

Fused heterocycles with chiral subunits for R¹ were synthesized (FIG. 3) to further explore their activity against wild-type CCRF CEM and multidrug-resistant P glycoprotein-overexpressing CEM/ADR5000 cells. Preliminary results indicated an influence of the structure of the α-carbon next to the thiourea group on the anti-cancer activity. Chiral quinazoline-thiohydantoin fused heterocycles with an unsubstituted quinazoline backbone were obtained with moderate to good yields (50-77%, 5n-5w) and even bulky residues like artemisinin were tolerated (5w). With the (R)-1-phenylethan residue for R¹ the influence of the variation of substituents R² on the quinazoline backbone was investigated (5a-5m). The yields of 5f and 5g were lower than the one of 5e. Product 5h with two electron withdrawing chlorine substituents had a low yield. Substrates with highly electronegative substituents like nitro (5d) and fluorine (5l and 5m) also gave low yields. It is assumed that lower electron density in the aromatic ring slows down the imine formation and lowers the basicity of the thiourea. As a result, the aza-Mannich reaction is hindered. Electron donating groups (5i-5k) had marginal influence on the yields. In summary, the variation of R² had less influence on the reaction yields than the variation of R¹ and electron withdrawing groups in R¹ and bulky groups in R² lowered the yield.

Some of the synthesized quinazoline-thiohydantoin fused heterocycles were tested against sensitive wild-type CCRF CEM and multidrug resistant P glycoprotein-overexpressing CEM/ADR5000 cells (Table 2). Doxorubicin was employed as a reference compound. the compounds of the present invention showed a good anti-leukemic effect. Indeed, the combination of the quinazoline-thiohydantoin-backbone with an artemisinin-moiety (5w) led to a hybrid-type structure with an IC₅₀ value of 0.165±0.019 μM. By comparing this result with the low activity of pure artemisinin (IC₅₀: 36.90±6.90 μM) and the very simple quinazoline-thiohydantoin fused heterocycles (3n) the power of the hybridization concept is revealed. The hybrid 5w is much more active than its building blocks. 5s and 5t show a similar anti-leukemia effect (5s: IC₅₀: 0.200±0.028 μM and 5t: IC₅₀=0.191±0.038 μM). 5u has an IC₅₀=0.518±0.046 μM. In addition, the substitution pattern of the quinazoline ring influences the anti-leukemia activity of the quinazoline-thiohydantoin fused heterocycles. The nitro— and chlorine-containing derivatives 5d, 5g and 5k showed remarkable influence on the cell viability of wild-type CCRF CEM. 5g had a high activity with an IC₅₀ value of 0.125±0.024 μM.

In addition to leukemia cell lines, the quinazoline-thiohydantoin fused heterocycles were applied against healthy cells to investigate their selectivity. Indeed, the tested compounds were significantly less active against healthy human cells, than against sensitive and multidrug resistant leukemia cells. For example, compounds 5d, 5f and 5g were highly active against multidrug resistant leukemia cells CEM/ADR5000 (IC₅₀=0.127±0.019, 0.156±0.013 and 0.146±0.015 μM, respectively) but in contrast to this they show a fifteen to twenty times lower activity against healthy cells (IC₅₀=2.5±0.8, 2.3±0.7 and 2.8±0.6 μM, respectively). Serum Response Factor (SRF) and its transcriptional coactivators MRTF-A and -B have been shown to trigger tumor growth (NPL-37 and NPL-38). Previous work from the present inventors revealed that constitutive nuclear localization of MRTF-A and -B prevails in hepatocellular and mammary carcinoma cells as well as in leukemia cells lacking the tumor suppressor deleted in liver cancer 1 (DLC1) (NPL-39 and NPL-40). Recent evidence implicated active nuclear MRTF-A as a dominant driver of tumor resistance and as a biomarker to predict tumor responsiveness to MRTF inhibitors (NPL-41). It was found that pharmacological inhibition of MRTF-A nuclear localization has antitumor effects by inducing oncogene-induced senescence (NPL-39).

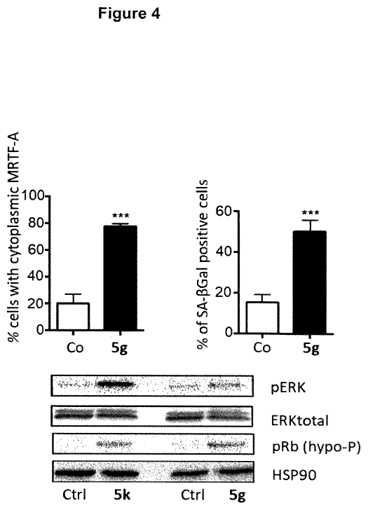

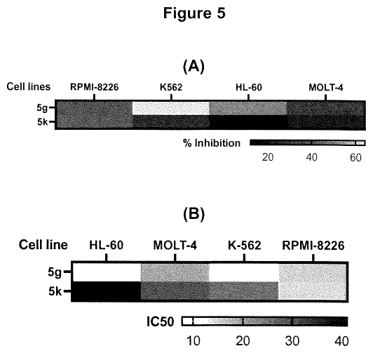

Quinazoline-thiohydantoin fused heterocycles 5g and 5k were to tested to determine whether they can inhibit MRTF-A nuclear localization to overcome resistance. First the localization of MRTF-A in a human leukemia cell line (HAP1) was analyzed. Treatment with 5g efficiently increased cytoplasmic MRTF-A nuclear localization. It was also tested whether 5g can activate cellular senescence. Indeed, 5g was able to provoke a senescence response mediated by the oncogene Ras and downstream MAPK/pRb pathways (C, FIG. 4). This data suggests that particularly 5g is a promising anti-leukemia agent for combatting drug resistance.

TABLE 2
IC₅₀ values for doxorubicin, artemisinin, achiral (3) and
chiral (5) quinazoline-thiohydantoin fused heterocycles in
sensitive wild-type CCRF CEM and multidrug resistant P
glycoprotein-overexpressing CEM/ADR5000 cells.

| | | | CEM/ | Healthy |
|---|---|---|---|---|
| | Molecular | CCRF-CEM | ADR5000 | cells |
| Com- | Weight | IC₅₀ | IC₅₀ | IC₅₀ |
| pound | (g/mol) | (μM) | (μM) | (μM) |
| Doxo- | 579.98 | 0.009^{[a]} | 23.27^{[a]} | 1.8^{[b]} |
| rubicin | | | | |
| Arte- | 282.14 | 36.90 ± 6.90^{[a]} | 26.90 ± 4.40^{[a]} | 69.56^{[c]} |
| misinin | | | | |
| 3a | 338.34 | 27.758 ± 2.596 | 26.820 ± 1.668 | 19.8 ± 4.8 |
| 3j | 323.37 | 28.092 ± 0.831 | 28.051 ± 0.972 | 42.8 ± 6.1 |
| 3k | 293.34 | n. a. | n. a. | n. a |
| 3l | 377.51 | 25.088 ± 6.493 | 19.139 ± 7.103 | 30.5 ± 0.5 |
| 3n | 245.30 | n. a. | n. a. | n. a |
| 3o | 259.33 | n. a. | n. a. | n. a |
| 3p | 305.41 | n. a. | n. a. | n. a |
| 5a | 400.29 | 0.483 ± 0.025 | 0.409 ± 0.028 | 2.2 ± 0.2 |
| 5d | 366.40 | 0.140 ± 0.028 | 0.127 ± 0.019 | 2.5 ± 0.8 |
| 5e | 355.84 | 0.375 ± 0.043 | 0.295 ± 0.083 | 2.4 ± 0.5 |
| 5f | 355.84 | 0.228 ± 0.022 | 0.156 ± 0.013 | 2.3 ± 0.7 |
| 5g | 355.84 | 0.125 ± 0.024 | 0.146 ± 0.015 | 2.8 ± 0.6 |
| 5k | 351.42 | 1.533 ± 0.148 | 1.332 ± 0.247 | 1.9 ± 0.4 |
| 5l | 335.42 | 0.516 ± 0.042 | 0.472 ± 0.051 | 1.5 ± 0.02 |
| 5n | 321.40 | 0.482 ± 0.025 | 0.460 ± 0.007 | 2.9 ± 0.2 |
| 5o | 321.40 | 0.516 ± 0.009 | 0.489 ± 0.016 | 5.9 ± 0.8 |
| 5p | 400.29 | 0.565 ± 0.104 | 0.532 ± 0.090 | 3 ± 0.7 |
| 5q | 273.35 | n. a. | n. a. | n. a |
| 5r | 289.35 | 11.247 ± 3.543 | 5.287 ± 0.668 | >100 |
| 5s | 371.46 | 0.200 ± 0.028 | 0.242 ± 0.024 | 2.1 ± 1.5 |
| 5t | 371.46 | 0.191 ± 0.038 | 0.322 ± 0.093 | 2.7 ± 1.3 |
| 5u | 371.46 | 0.518 ± 0.046 | 0.525 ± 0.034 | 3.1 ± 0.01 |
| 5v | 347.44 | 25.053 ± 9.151 | 12.932 ± 3.563 | 37.9 ± 1.5 |
| 5w | 525.66 | 0.165 ± 0.019 | 0.176 ± 0.034 | >10 |

^{[a]}EC₅₀ values for sensitive wild-type CCRF CEM and multidrug resistant P glycoprotein-overexpressing CEM/ADR5000 cells have been previously reported for doxorubicin (NPL-9) and artemisinin (NPL-10).
^{[b]}In normal tissue cell line (human fibroblasts). DOX and DOX-TRF were tested to obtain an overall IC₅₀ of 1.8 μM for the free drug and 2.8 μM for the conjugate (NPL-11).
^{[c]}The IC₅₀ values for NIH-3T3 cells and human endometrium cells were 105.77 μM or 69.56 μM for artemisinin (NPL-12).

Apart from the above presented results, chiral quinazoline-thiohydantoin fused heterocycles (5a-5w) demonstrated an impressive influence on the cell viability of multidrug resistant P glycoprotein-overexpressing CEM/ADR5000 cells. All tested chiral compounds showed higher anti-leukemia activity than doxorubicin (IC₅₀=23.27 μM), which is currently used for conventional chemotherapy. The activity against wild-type CCRF CEM cells of the naphthalene-containing derivatives 5s, 5t and 5u is slightly lower than against multidrug resistant P glycoprotein-overexpressing CEM/ADR5000 cells, but still in the same range (5s: IC₅₀=0.242±0.024 μM, 5t: IC₅₀=0.191±0.038 μM and 5u: IC₅₀=0.525±0.034). Also, the artemisinin-derived hybrid compound 5w can overcome the multidrug resistance of leukemia cells. It shows an IC₅₀ value of 0.176±0.034 μM. Even higher activity against the described cell line was measured for the chlorine-containing derivatives 5f (IC₅₀=0.156±0.013 μM) and 5g (IC₅₀=0.146±0.015). The position of the substituent on the aromatic ring of the quinazoline seems to substantiate the difference of their anti-leukemia activity. With an IC₅₀ value of 0.127±0.019 μM the nitro-derived derivative 5d showed the highest activity of all tested compounds against multidrug resistant P glycoprotein-overexpressing CEM/ADR5000 cells. This outstanding result proves the great potential of the quinazoline-thiohydantoin core structure as a building block of prospective anti-leukemia agents to overcome multidrug resistance. Achiral structures (3a-3p) showed no significant activity against leukemia cells, neither CCRF CEM cell line nor CEM/ADR5000 cell line.

To investigate the spectrum of the compounds 5g & 5k, both of the compounds were tested on four leukemia cells at one dose (10 μM) (FIG. 5A). It was found that 5g was more active compared to 5k in K562 and HL-60 cell lines, as co. To further calculate the IC₅₀ value of compounds 5g and 5k, a cell viability assay was performed at five different doses (FIG. 5B). The results of this analysis are shown in the following table. The data suggests that compound 5g has a better anti-cancer activity against leukemia cells, as compared to 5k.

TABLE
IC₅₀ values of 5g and 5k

| Compd/Cells | RPMI-8226: | K562: | HL-60 | MOLT-4 |
|---|---|---|---|---|
| 5g | 15 ± 1.39 | 7.8 ± 0.63 | 9.9 ± 0.71 | 18.5 ± 2.17 |
| 5k | 14 ± 2.43, | 24.7 ± 4.01 | 41.1 ± 3.12 | 28.7 ± 2.08 |

### Cell Lines

All leukemia cell lines (RPMI-8226, K562, HL-60, and MOLT-4) were obtained from NCI-DTP, NCI-Frederick, USA. Cells were cultured and maintained in RPMI-1640 (Corning, USA; #10-040-CV) media supplemented with 10% FBS (Corning, USA; #35-015-CV) and 1% penicillin-streptomycin-amphotericin B (GIBCO, USA; #15240-062) at 37° C. with 5% CO₂. At 70 to 80% confluence cells were harvested and plated for cell viability assay.

### Cell Viability Assay

Each leukemia cell line was plated at 3000 cells/well (in 100 μL complete media) in black-clear bottom 96-well plates (Corning, USA; #3603). After 24 h, cells were treated with respective compounds at one dose (10 μM) or five dose (0.01, 0.1, 1, 10, 100 μM) concentrations. After 48 h treatment, cell viability was recorded using CellTiter-Blue cell viability assay (Promega, USA; #G8081) as per the manufacturer's protocol.

1. A compound having the formula (I) 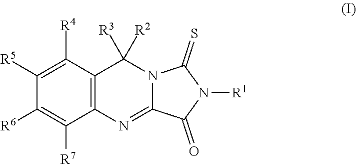
a stereoisomer, racemic mixture, tautomer, pharmaceutically acceptable salt, prodrug, hydrate, solvate, or polymorph thereof;
wherein
R¹ is selected from alkyl, alkenyl, alkinyl, a carbocyclic moiety, a heterocyclic moiety, an -L-carbocyclic moiety, and an -L-heterocyclic moiety, wherein the alkyl, alkenyl, and alkinyl can optionally contain one or more catenary oxygen, nitrogen or sulfur atoms;
R² and R³ are independently selected from H, alkyl, alkenyl, alkinyl, a carbocyclic moiety, a heterocyclic moiety, an -L-carbocyclic moiety, and an -L-heterocyclic moiety;
R⁴, R⁵, R⁶, and R⁷ are independently selected from H, alkyl, alkenyl, alkinyl, a carbocyclic moiety, a heterocyclic moiety, an -L-carbocyclic moiety, an -L-heterocyclic moiety, halogen, —NO₂, —O—R⁸, —S—R⁸, —CN, —OCN, —SCN, —NCS, —N₃ and —NR^{a}R^{b};
R⁸ is independently selected from H, alkyl, alkenyl, alkinyl, a carbocyclic moiety, and a heterocyclic moiety;
R^{a} and R^{b} are independently selected from H, alkyl, a carbocyclic moiety, and a heterocyclic moiety;
L is independently selected from an alkylene moiety, the chain of which can be optionally interrupted by one or more —O—, —S—, —N(R)—, —C(O)—, —C(O)—O—, —N(R)—C(O)—O—, and —C(O)—N(R)—;
R is independently selected from H and alkyl;
wherein the alkyl, alkenyl, alkinyl, alkylene, carbocyclic moiety and heterocyclic moiety can be optionally substituted.

2. The compound according to claim 1, wherein
R¹ is selected from an -L-carbocyclic moiety and an -L-heterocyclic moiety,
L is independently selected from an alkylene moiety, the chain of which can be optionally interrupted by one or more —O—; and
wherein the alkylene, carbocyclic moiety and heterocyclic moiety can be optionally substituted.

3. The compound according to claim 2, wherein
R¹ is selected from an -L-aryl,
L is independently selected from an alkylene moiety, the chain of which can be optionally interrupted by one or more —O—; and
wherein the aryl can be optionally substituted.

4. The compound according to claim 3, wherein R¹ is selected from —C(H)(CH₃)-aryl, wherein the aryl moiety can be optionally substituted.

5. The compound according to claim 1, wherein R⁴, R⁵, R⁶, and R⁷ are independently selected from H, halogen, NO₂, —OH, and —O-alkyl, in particular wherein one or two of R⁴, R⁵, R⁶, and R⁷ is independently selected from halogen, —NO₂, —OH, and —O-alkyl and the others of R⁴, R⁵, R⁶, and R⁷ are H.

6. The compound according to claim 1, wherein R² and R³ are both H.

7. The compound according to claim 1, wherein R¹ is selected from a carbocyclic moiety, a heterocyclic moiety, an -L-carbocyclic moiety, and an -L-heterocyclic moiety, preferably R¹ is selected from an -L-carbocyclic moiety, and an -L-heterocyclic moiety, wherein the carbocyclic moiety and the heterocyclic moiety are selected from a pharmacophore.

8. A pharmaceutical composition comprising a compound according to claim 1, a stereoisomer, racemic mixture, tautomer, pharmaceutically acceptable salt, prodrug, hydrate, solvate, or polymorph thereof; and optionally a pharmaceutically acceptable carrier.

9. (canceled)

10. A method of treating, ameliorating or preventing a proliferative disorder comprising administering a therapeutically effective amount of a compound according to claim 1, a stereoisomer, racemic mixture, tautomer, pharmaceutically acceptable salt, prodrug, hydrate, solvate, or polymorph thereof to a patient in need thereof, wherein the proliferative disorder is preferably leukemia, breast cancer, skin cancer, lung cancer, human embryonic kidney cells, or cervical cancer, more preferably leukemia.

11. A method of producing a compound according to claim 1, the method comprising:
(i) providing a compound having formula (II) 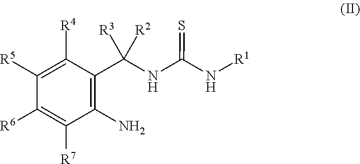
(ii) reacting the compound having the formula (II) with a compound having the formula (III) 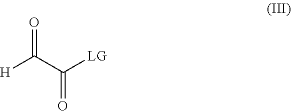
wherein R¹, R², R³, R⁴, R⁵, R⁶, and R⁷ are as defined in claim 1; and LG is a leaving group

12. The method according to claim 11, wherein the leaving group LG is selected from —O-alkyl, —OH, —O-aryl, -halogen, —O—SO₂CF₃, tosylates, and mesylates, preferably —O-alkyl.

13. The method according to claim 11, wherein the reaction between the compound having the formula (II) and the compound having the formula (III) is conducted by a domino reaction followed by a dehydrogenation reaction.

14. The method according to claim 13, wherein the domino reaction is conducted in the presence of a Brønsted acid, which is preferably selected from trifluoroacetic acid, phosphoric acid diesters, acetic acid, and benzoic acid, more preferably trifluoroacetic acid.

15. The method according to claim 13, wherein the dehydrogenation reaction is carried out in the presence of an oxidant, which is preferably selected from air, oxygen, MnO₂, 2,3-dichloro-5,6-dicyano-1,4-benzoquinone and the combination of hydrogen peroxide-urea and iodine more preferably 2,3-dichloro-5,6-dicyano-1,4-benzoquinone.

16. The method according to claim 11, wherein the method is a one-pot reaction.
